(19) United States
(12) Patent Application Publication
(10) Pub. No.: US 2025/0262112 A1
(43) Pub. Date: Aug. 21, 2025

(54) FOREARM EXOSKELETON FOR TREMOR ALLEVIATION

(71) Applicant: VIRGINIA TECH INTELLECTUAL PROPERTIES, INC., Blacksburg, VA (US)

(72) Inventors: JIAMIN WANG, Blacksburg, VA (US); OUMAR BARRY, Blacksburg, VA (US)

(21) Appl. No.: 18/854,368

(22) PCT Filed: Apr. 28, 2023
(86) PCT No.: PCT/US2023/066384

§ 371 Date: Oct. 4, 2024

Related U.S. Application Data
(60) Provisional application No. 63/363,876, filed on Apr. 29, 2022.

(51) Int. Cl. A61H 1/02

(52) U.S. Cl. CPC A61H 1/0285; A61H 2201/0107; A61H 2201/1207; A61H 2201/1635; A61H 2201/165; A61H 2201/1659; A61H 2201/1676; A61H 2201/5007; A61H 2201/5064; A61H 2230/605

(57) ABSTRACT

The embodiments described herein are directed to a non-invasive wearable forearm exoskeleton that provides condition monitoring, tremor alleviation, and movement assistance at the human forearm joints in activities of daily living. The rigid linkages in the exoskeleton can adapt to different forearm profiles, allowing the user to perform natural forearm motions and transmit forces and torques efficiently. The exoskeleton can be worn by the user comfortably through soft interfacing. Encoders and inertia measurement units provide accurate measurements of the forearm motions. Through signal processing, a tremor signal is separated from voluntary motion for assessment and control. The motors produce safe actuations based on adaptive control and motion planning algorithms that simultaneously suppress tremors and assist voluntary motions.

### CROSS-REFERENCE TO RELATED APPLICATIONS

This application claims the benefit of and priority to U.S. Provisional Application Ser. No. 63/363,876, titled “Forearm Exoskeleton For Tremor Alleviation,” filed Apr. 29, 2022, the entire contents of which is hereby incorporated herein by reference.

### BACKGROUND

A tremor is an involuntary quivering movement or shake in the limbs of the body of an individual. Tremors are common in individuals afflicted with Parkinson's disease, among other conditions. The slow, rhythmic tremor of Parkinson's disease can start in one hand, foot, or leg and can eventually affect both sides of the body. The resting tremor of Parkinson's disease can also occur in the jaw, chin, mouth, or tongue. Some people with Parkinson's disease can also experience a feeling of internal tremor. Tremors can be particularly frustrating for motor planning. Tremors also attract attention, and individuals with Parkinson's disease tend to keep tremoring hands in pockets.

### SUMMARY OF THE INVENTION

The embodiments described herein are directed to a non-invasive wearable forearm exoskeleton that provides condition monitoring, tremor alleviation, and movement assistance at the human forearm joints in activities of daily living. The rigid linkages in the exoskeleton can adapt to different forearm profiles, allowing the user to perform natural forearm motions and transmit forces and torques efficiently. The exoskeleton can be worn by the user comfortably through soft interfacing. Encoders and inertia measurement units provide accurate measurements of the forearm motions. Through signal processing, a tremor signal is separated from voluntary motion for assessment and control. The motors produce safe actuations based on adaptive control and motion planning algorithms that simultaneously suppress tremors and assist voluntary motions.

In one example, a forearm exoskeleton for an arm includes a plurality of attachment modules, a plurality of pivotable linkages between the plurality of attachment modules, a plurality of motors mechanically coupled between the attachment modules and the pivotable linkages, feedback sensors that provide position feedback for the exoskeleton, and a controller configured to direct one or more of the motors to suppress tremors in and assist with voluntary motions of the arm based on the position feedback. The pivotable linkages can include a first link, a second link, a pivot point between the first link and the second link, and a position encoder at the pivot point in one example.

In other aspects, the controller is further configured to direct one or more of the motors of the forearm exoskeleton based on one or more kinematic models for the arm, one or more kinematic models for the forearm exoskeleton, and a motion planning algorithm using the position feedback and the models. The controller is also configured to isolate involuntary movement from voluntary movement in the arm based on a frequency analysis performed on the position feedback. The controller is also configured to direct the motors of the forearm exoskeleton based on a motion planning algorithm to suppress the involuntary movement and assist with the voluntary movement in the arm.

The feedback sensors can include at least one inertial measurement unit, at least one positional encoder in at least one of the plurality of motors, and at least one positional encoder in at least one of the plurality of pivotable linkages. At least one pivotable linkage among the plurality of pivotable linkages can include an Euler-type joint. The attachment modules can include an attachment module for a bicep of the arm, an attachment module for a proximal end of a forearm of the arm, an attachment module for a distal end of the forearm, and an attachment module for a dorsum of a hand of the arm.

In other aspects, the controller is also configured to perform a first kinematic identification algorithm for elbow flexion-extension (EFE) motion of the arm using the position feedback, perform a second kinematic identification algorithm for forearm pronation-supination (FPS) motion of the arm using the position feedback, perform a third kinematic identification algorithm for wrist flexion-extension (WFE) motion of the arm using the position feedback, and perform a fourth kinematic identification algorithm for wrist radial-ulnar deviations (RUD) motions of the arm using the position feedback. The controller can develop a model for the arm based on a combination of the first, the second, the third, and the fourth kinematic identification algorithms, and direct the motors based on the model for the arm using the position feedback. The controller is also configured to recursively update the models for the arm, the models for the forearm exoskeleton, or both in real-time.

In other examples, the controller is also configured to perform trajectory tracking of an intended motion of the arm based on the position feedback, forecast a trajectory of the intended motion of the arm based on the position feedback, and direct the plurality of motors based on a motion planning algorithm to suppress involuntary movement in the arm and to assist with voluntary movement in the arm based on the forecast of the trajectory of the intended motion.

In another example, a method for control of a forearm exoskeleton for an arm includes analyzing movement data of the forearm exoskeleton based on position feedback from feedback sensors of the forearm exoskeleton, generating a model for the forearm exoskeleton based on the analyzing, planning motion for the arm and the exoskeleton based on the model, and directing at least one motor of the forearm exoskeleton based on the motion planning.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates an example forearm exoskeleton for tremor alleviation according to various embodiments described herein.

FIG. 2 illustrates a top-down view of the forearm exoskeleton shown in FIG. 1 according to various embodiments described herein.

FIG. 3 illustrates a side view of the forearm exoskeleton shown in FIG. 1 according to various embodiments described herein.

FIG. 4 illustrates example pivot points of the pivotable linkage in the exoskeleton shown in FIG. 1 according to various embodiments described herein.

FIG. 5 illustrates example pivot points of another pivotable linkage in the exoskeleton shown in FIG. 1 according to various embodiments described herein.

FIG. 6 illustrates example pivot points of another pivotable linkage in the exoskeleton shown in FIG. 1 according to various embodiments described herein.

FIG. 7 illustrates an example method of tremor alleviation and voluntary movement assistance using an exoskeleton according to various embodiments described herein.

### DETAILED DESCRIPTION

The embodiments described herein are directed to a non-invasive wearable forearm exoskeleton that provides condition monitoring, tremor alleviation, and movement assistance at the human forearm joints in activities of daily living. The 6-degree-of-freedom (DOF) rigid linkages can adapt to different forearm profiles, allowing the user to perform natural forearm motions and transmit forces and torques efficiently. The exoskeleton is worn by the user comfortably through soft interfacing. Encoders and inertia measurement units provide accurate measurements of forearm motions. Through signal processing, the tremor signal is separated from voluntary motion for assessment and control. The servo motors produce safe actuations based on adaptive control and motion planning algorithms that simultaneously suppress tremors and assist voluntary motions.

The exoskeleton embodiments described herein do not require skin-contacting sensors (e.g., EMG sensors, Goniometers, etc.) for control. Encoders and inertia measurement units in the exoskeleton provide accurate measurements of forearm motions. Through signal processing, feedback data representative of tremors can be separated from voluntary motions by a control system of the exoskeleton, for assessment and control. A number of motors in the exoskeleton produce safe actuations based on adaptive control and motion planning algorithms that simultaneously suppress tremors and assist with voluntary motions. Overall, the embodiments aim to provide non-invasive, compliant, and stable tremor rehabilitation, as well as movement assistance at the forearm for individuals experiencing tremors. The embodiments are also applicable to reduce fatigue in manual laborers tasked with repetitive and dexterous tasks through continual assistance, among other uses.

The exoskeleton embodiments described herein can actively suppress tremors for the elbow flexion-extension (EFE), the forearm pronation-supination (FPS), the wrist flexion-extension (WFE), and the wrist radial-ulnar deviations (RUD) motion in the arm. The exoskeleton includes rigid linkage mechanisms extending between attachment modules located at the lateral side of upper arm or bicep, the proximal end of forearm, the distal end of the forearm, and the dorsum of the hand, respectively. The attachment modules can be adapted (e.g., sized or fitted) to different bicep, forearm, wrist, and hand profiles of users and allow fully natural arm movements. The motors in the exoskeleton can provide a wide range of actuations or movements of the arm. For tremor suppression and partial movement assistance, the maximum torques of the motors are approximately 2 N-m for the WFE and RUD motions, and 4 N-m for the WPS and EFE motions in one example. However, the motors can provide other maximum torques in other cases, depending on various factors such as the desired power efficiency and loading safety of the exoskeleton.

To improve ergonomics (e.g., non-invasiveness, compliance, and stability) of the design, the exoskeleton embodiments described herein can be worn using soft and non-binding interfaces, including gloves, arm bands, and hook and loop fastener strips. The battery and controller of the exoskeleton can be located at the upper arm to reasonably distribute weight. Cables run through the mechanisms to avoid entanglements. The sensors of the exoskeleton include IMUs and encoders, which provide full measurements of forearm kinematics as described herein. The controller employs advanced techniques in signal processing, robotics, and motion planning to ensure safe and compliant operations. A touch-screen user interface can also be coupled, either by wired or wireless communications, to directly configure the settings of the exoskeleton. The controller can also include fail-safe systems that detect anomalies, such as electric malfunctions and hardware damage.

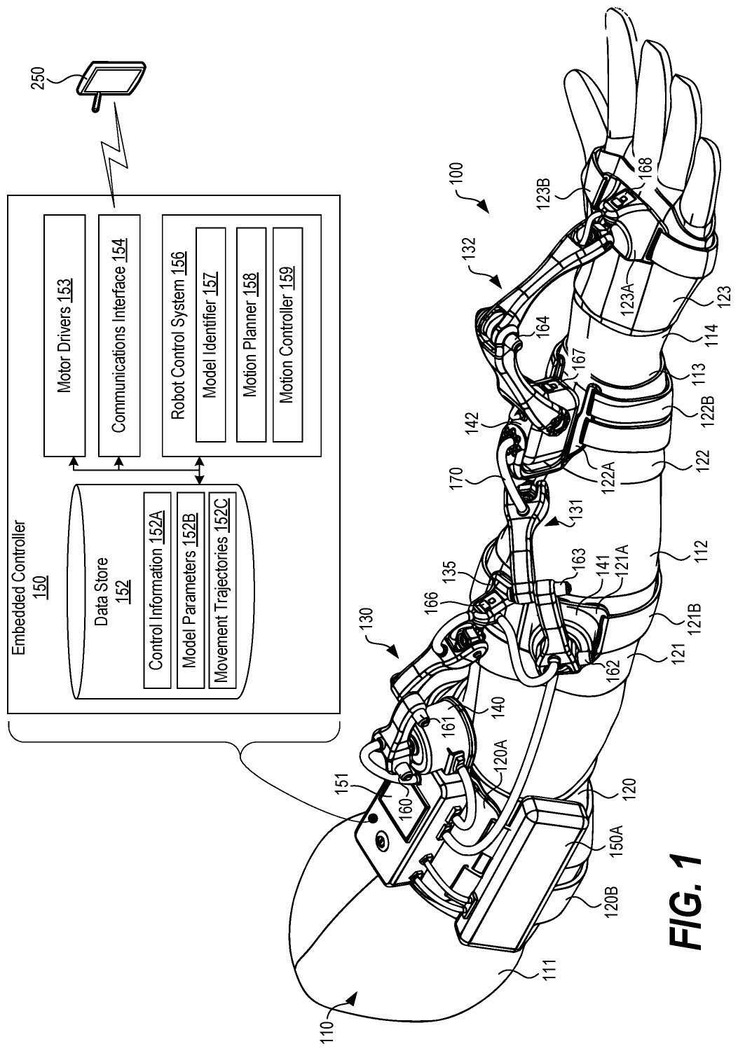

FIG. 1 illustrates an example forearm exoskeleton 100 for tremor alleviation according to various embodiments described herein. The exoskeleton 100 is illustrated as a representative example of one type of system that can help an individual with both tremor suppression and to completely voluntary, intended motions. The tremor suppression and motion assistance concepts described herein are not limited to use with any particular type of exoskeleton or related device. The exoskeleton 100 is not exhaustively illustrated and may include other features or components that are not shown. In some cases, one or more of the components of the exoskeleton 100, as shown and described below, can be omitted. Additionally, the concepts described herein can be extended to exoskeletons and related devices that can be worn or fitted to the leg, neck, torso, or other areas of the body.

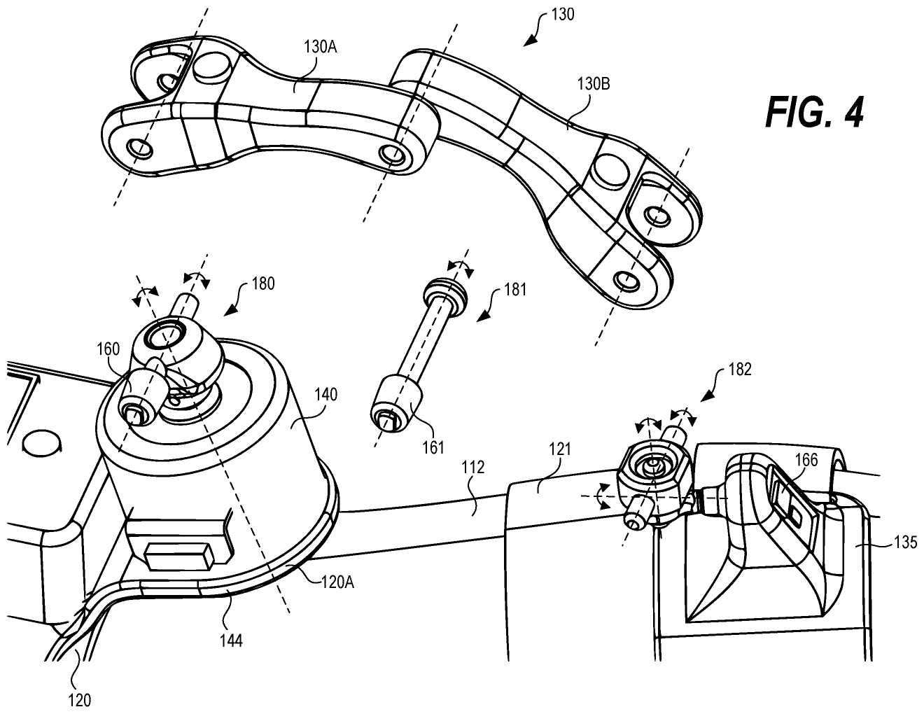

The exoskeleton 100 is shown attached to an arm 110 of an individual in FIG. 1. Among other components, the exoskeleton 100 includes attachment modules 120-123 for attachment to the arm 110, attachments platforms 120A-123A and straps 120B-123B of the attachment modules 120-123, pivotable linkages 130-132 between the attachment modules 120-123, attachment anchors 135 and 136, and motors 140-142 mechanically coupled between the attachment modules 120-123 and the pivotable linkages 130-132. The exoskeleton 100 also includes an embedded controller 150, a battery 150A, a number of signaling and power cables, such as the cable 170, positional encoders 160-164, inertial measurement units (IMUs) 166-168, and other components described herein. The cable 170 can be routed through the pivotable linkages 130-132 as shown in FIG. 1 to avoid entanglements. Additional components of the exoskeleton 100 are also illustrated in FIGS. 4-6.

The exoskeleton 100 provides both active and passive suppression of tremors or involuntary movement in the arm 110. As for passive suppression of involuntary movement, the kinematic chain formed by the motors 140-142 and the pivotable linkages 130-132 between the attachment modules 120-123 is elastic and provides resistance against involuntary motion to some extent, even without model-based control of the motors 140-142. As for active suppression of involuntary movement, the motors 140-142 are directed by the motor drivers 153 and the robot control system 156 to suppress (e.g., counterbalance against) the involuntary movements of the arm 110. Additionally, the exoskeleton 100 provides active assistance to the voluntary movement of the arm 110. These and other aspects of the exoskeleton 100 are described below.

The attachment modules 120-123 can be formed from any suitable materials, including fabric, rubber, neoprene, plastic, other materials, and combinations thereof. The attachment modules 120-123 can be formed to any suitable size and shape depending on the size of the arm to which the exoskeleton 100 is fitted. The attachment modules 120-123 can also be held in place, in part, by bands, strips of hook and loop fastener, ties, or other means as described below.

More particularly, the attachment module 120 can include a sleeve formed from a flexible fabric, rubber, neoprene, plastic, other related material, or combinations thereof, sized to fit around the bicep 111 of the arm 110. The attachment module 120 can be formed to any suitable size and shape depending on the size of the bicep 111. The attachment module 120 includes the attachment platform 120A. The attachment platform 120A can be formed from a more rigid material, such as a firm plastic or metal material. The motor 140 can be secured to a surface region of the attachment platform 120A using adhesives, mechanical fasteners, mechanical interferences or interlocks, other attachment means, or combinations thereof. A number of straps, such as the strap 120B, extend through mounting apertures in the attachment platform 120A and around the sleeve of the attachment module 120 and the bicep 111. The strap 120B can be embodied as an elastic strap and, in some cases, include one or more fasteners, such as hook-and-loop fasteners, hooks, ties, or other means, to secure the attachment platform 120A around the sleeve and hold the attachment module 120 in place around the bicep 111.

The attachment module 121 can include a sleeve formed from a flexible fabric, rubber, neoprene, plastic, other related material, or combinations thereof, sized to fit around the forearm 112 of the arm 110. The attachment module 121 can be formed to any suitable size and shape depending on the size of the forearm 112. The attachment module 121 includes the attachment platform 121A. The attachment platform 121A can be formed from a more rigid material, such as a firm plastic or metal material. The motor 141 can be secured to a first region of the attachment platform 121A using adhesives, mechanical fasteners, mechanical interferences or interlocks, other attachment means, or combinations thereof. The attachment anchor 135 can be formed integrally with or be secured to another region of the attachment platform 121A. The attachment anchor 135 includes a joint for interfacing with one end of the pivotable linkage 130. A number of straps, such as the strap 121B, extend through mounting apertures in the attachment platform 121A and around the sleeve of the attachment module 121 and the forearm 112. The strap 121B can be embodied as an elastic strap and, in some cases, include one or more fasteners, such as hook-and-loop fasteners, hooks, ties, or other means, to secure the attachment platform 121A around the sleeve and hold the attachment module 121 in place around the forearm 112.

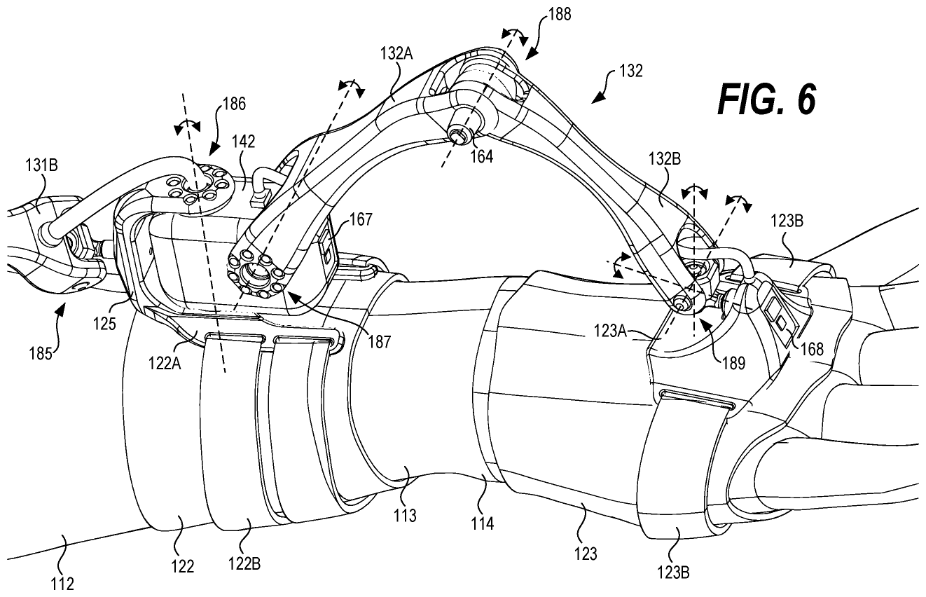

The attachment module 122 can include a sleeve formed from a flexible fabric, rubber, neoprene, plastic, other related material, or combinations thereof, sized to fit around the wrist 113 of the arm 110. The attachment module 122 can be formed to any suitable size and shape depending on the size of the wrist 113. The attachment module 122 includes the attachment platform 122A. The attachment platform 122A can be formed from a more rigid material, such as a firm plastic or metal material. A hub of the motor 142 can be secured to the attachment platform 122A, as described in further detail below with reference to FIG. 6. A number of straps, such as the strap 122B, extend through mounting apertures in the attachment platform 122A and extend around the sleeve of the attachment module 122 and the wrist 113. The strap 122B can be embodied as an elastic strap and, in some cases, include one or more fasteners, such as hook-and-loop fasteners, hooks, ties, or other means, to secure the attachment platform 122A around the sleeve and hold the attachment module 122 in place around the wrist 113.

The attachment module 123 can include a glove, possibly with open fingers, formed from a flexible fabric, rubber, neoprene, plastic, other related material, or combinations thereof, sized to fit around the hand 114 of the arm 110. The attachment module 123 can be formed to any suitable size and shape depending on the size of the hand 114. The attachment module 123 includes the attachment platform 123A, which can be formed from a more rigid material, such as a firm plastic or metal material. The attachment platform 123A includes a joint for interfacing with one end of the pivotable linkage 132. A number of straps, such as the strap 123B, extend through mounting apertures in the attachment platform 123A and around the glove of the attachment module 123. The strap 123B can be embodied as an elastic strap and, in some cases, include one or more fasteners, such as hook-and-loop fasteners, hooks, ties, or other means, to secure the attachment platform 123A around the glove and hold the attachment module 123 in place around the hand 114.

The motors 140-142 are secured to the rigid platforms of the attachment modules 120-123. The motors 140-142 can be embodied as any suitable motors, such as servo motors, brushless motors, or other motors, and the motors 140-142 can incorporate positional encoders in some cases. The motors 140 and 141 include a single motor shaft and degree of freedom with respect to the shaft. The motor 142 includes two separate motors. The motors 140-142 are mechanically coupled to other components in the kinematic chain of the exoskeleton 100 at a number of joints, as described in detail below with reference to FIGS. 4-6. The motors 140-142 can be powered and driven by the motor drivers 153 in the embedded controller 150 at the direction of the robot control system 156, as described herein.

The motors 140-142 can incorporate positional encoders. Thus, the motors 140-142 can provide feedback signals or data to the embedded controller 150, with the feedback signals identifying the absolute or relative positions of the shafts or other internal moving parts of the motors 140-142. As described in further detail below, the embedded controller 150 can calculate information related to the positions, orientations, and movement of the attachment modules 120-123 and the pivotable linkages 130-132 of the exoskeleton 100 based in part on the feedback signals from the motors 140-142. The embedded controller 150 can also calculate information related to the position of the arm 110, including the positions and orientations of the bicep 111, the forearm 112, the wrist 113, and the hand 114 of the arm 110.

The arm 110 may experience involuntary quivering movement due to Parkinson's disease, for example. The arm 110 may also move, at least in part, in an intended direction or directions based on the intentions of an individual. The embedded controller 150 is configured to analyze and monitor the motion of the arm 110 based on the feedback signals and data provided from the positional encoders in the motors 140-142, the positional encoders 160-164, and the IMUs 166-168 (also referenced herein collectively as “feedback sensors”). More particularly, the embedded controller 150 is configured to identify, isolate, and analyze the portion of the movement in the arm 110 that is attributed to involuntary quivering movements or shakes (e.g., the tremor motion) through a frequency or related analysis of the feedback data. The embedded controller 150 is also configured to identify, isolate, and analyze the portion of the movement in the arm 110 that is attributed to the intended or voluntary movements of the individual through a frequency or related analysis of the feedback data. The embedded controller 150 can also calculate and determine the trajectory of the intended motion of the arm 110 based in part on the feedback signals.

Based on the analysis of the feedback data, the embedded controller 150 is configured to direct the motors 140-142 to help suppress the involuntary movements in the arm 110 while, at the same time, direct the motors 140-142 to assist with the intended motion of the arm 110. Thus, the embedded controller 150 is capable of separately identifying the involuntary and voluntary movements in the arm 110. The embedded controller 150 can also process or execute adaptive control algorithms to learn the intended movements or motions of the individual and the arm 110 over time. These and other aspects of the embedded controller 150 are described below.

The pivotable linkages 130-132 provide mechanical linkages between the attachment modules 120-123 and the motors 140-142. The pivotable linkages 130-132 include one or more links and joints, as described in further detail below. The motors 140-142 can provide external forces to the pivotable linkages 130-132, both to aid in the intended movement of the arm 110 and to dampen involuntary motions or tremors in the arm 110. The pivotable linkages 130-132 can be formed from plastic, metal, other suitable materials, and combinations thereof. Examples of the links and pivot points or joints in the pivotable linkages 130-132 are described in further detail below with reference to FIGS. 4-6.

The positional encoders 160-164 are fitted and positioned at pivot points in the pivotable linkages 130-132. The positional encoders 160-164 can be embodied as angular position encoders. The positional encoders 160-164 can provide feedback signals to the embedded controller 150, with the feedback signals identifying the absolute or relative positions (e.g., angular positions) of links in the pivotable linkages 130-132 with respect to other links in the pivotable linkages 130-132, with respect to shafts of the motors 140-142, with respect to the rigid platforms in the attachment modules 120-123, or with respect to other points or frames of reference in the exoskeleton 100.

The embedded controller 150 can calculate information related to the positions, orientations, and movement of the attachment modules 120-123 and the pivotable linkages 130-132 of the exoskeleton 100 based in part on the feedback signals from the motors 140-142, the positional encoders 160-164, and the IMUs 166-168. The embedded controller 150 can also calculate information related to the position of and movement in the arm 110, including the positions, orientations, and movements of the bicep 111, the forearm 112, the wrist 113, and the hand 114 of the arm 110 based in part on the feedback data. The embedded controller 150 can also calculate, determine, and monitor the motion of the arm 110 based in part on the feedback signals. The embedded controller 150 is configured to direct the motors 140-142 to help suppress the involuntary movements in the arm 110 while, at the same time, direct the motors 140-142 to assist with the intended motion of the arm 110.

The embedded controller 150 can be embodied as a controller or microcontroller system. The embedded controller 150 can include one or more processors, one or more memory devices, and one or more motor driver circuits, among other components. The embedded controller 150 can be embodied as an embedded single-board microcontroller processing system, such as a Raspberry Pi, Arduino, or related single-board microcontroller. The embedded controller 150 can also be embodied as a type of programmable logic controller (PLC) system. The embedded controller 150, however, is not limited to any type or style of controller or microcontroller system. The embedded controller 150 is configured to receive and store data, such as the feedback data described herein. The embedded controller 150 is also configured to execute computer-readable code or instructions (e.g., software) to develop the models described below, to direct the operation of the exoskeleton 100, and to communicate or interface with other devices in some cases. These and other aspects of the embedded controller 150 are described in further detail below.

As shown in FIG. 1, the embedded controller 150 includes a data store 152. The data store 152 stores data for use and execution by the one or more processors of the embedded controller 150, including the control information 152A, the model parameters 152B, the motion trajectories 152C, and other data. The embedded controller 150 also includes motor drivers 153, a communications interface 154, and a robot control system 156 in the example shown. The robot control system 156 includes a model identifier 157, a motion planner 158, and a motion controller 159. The operation of the robot control system 156 is described in additional detail below.

In the data store 152, the control information 152A includes the feedback data provided by the motors 140-142, the positional encoders 160-164, and the IMUs 166-168, among feedback data provided from other sensors and systems of the exoskeleton 100. The control information 152A can be organized and stored in any suitable format for evaluation and processing by the robot control system 156. For example, the feedback data provided by the motors 140-142 can be stored separately from the feedback data provided by the positional encoders 160-164 and the IMUs 166-168. Similarly, the feedback data provided by the positional encoders 160-164 can be stored separately from that provided by the IMUs 166-168. The feedback data from the motors 140-142, the positional encoders 160-164, and the IMUs 166-168 can also be correlated or organized together in the data store 152. Additionally, the control information 152A can be correlated, organized, and processed in connection with the model parameters 152B, as described below.

The model parameters 152B include data related to the mechanical structure of the exoskeleton 100, such as the size of the exoskeleton 100, the positions of the attachment modules 120-123 on the arm 110, the characteristics (e.g., types, forces applied by) of the motors 140-142, the degrees of freedom in the pivot points or joints in the exoskeleton 100, the mechanical arrangements of the pivotable linkages 130-132, and other mechanical characteristics of the exoskeleton 100. The model parameters 152B can be organized and stored in any suitable format for evaluation and processing by the robot control system 156. In addition to feedback and model parameter data, the data store 152 can also store executable code (e.g., executable software) for execution by the one or more processors of the embedded controller 150. The executable code can be relied upon to implement the robot control system 156, for example, among other functional components of the embedded controller 150.

The motion trajectories 152C include data related to the movement of the arm 110. The motion trajectories 152C can include a historical database of the movements undertaken by the arm 110 during the performance of various tasks. The motion trajectories 152C can also include forecasted or estimated trajectories of the arm 110, as those forecasted trajectories are generated by the motion planner 158. The historical and forecasted motion trajectories 152C can be maintained in any suitable format with reference to any suitable coordinate or dimensionality reference or system. The motion trajectories 152C can also be developed in connection with kinematic models of the exoskeleton 100, kinematic models of the arm 110, and other models. The kinematic models of the exoskeleton 100 may be defined, in part, based on the model parameters 152B. The kinematic models of the arm 110 can be defined based on evaluations of the arm 110 performed by the model identifier 157, as described below.

The embedded controller 150 can also provide a type of user interface for the exoskeleton 100. To that end, the embedded controller 150 can include a user interface 151, which can be embodied as a capacitive touch screen or other type of display. A user of the exoskeleton 100 can interface with the user interface 151 to direct certain operations of the exoskeleton 100. In some cases, the embedded controller 150 can also include additional input/output (I/O) interfaces, such as one or more buttons, indicator lights, speakers, haptic feedback systems, and related I/O peripherals.

The motor drivers 153 can be embodied as semiconductor motor drivers for the motors 140-142. Based on control commands, signals, or other direction provided by the robot control system 156, the motor drivers 153 can provide power from the battery 150A to power and control the operation (e.g., the movement) of the motors 140-142 and the pivotable linkages 130-132 between the attachment modules 120-123, respectively.

The communications interface 154 can be embodied as one or more wired or wireless physical layer communications devices, such as a serial or parallel wired interface, a Bluetooth®, WiFi®, or other wireless interface, or a related data communications interface. The embedded controller 150 can interface with one or more external devices, such as the device 250, using the communications interface 154. Examples of the device 250 include those in the form of a desktop computer, laptop computer, personal digital assistant, cellular telephone, tablet computer, or other related computing device or system. The device 250 can be used to interface with the exoskeleton 100. For example, a user of the device 250 can monitor and control certain operations of the exoskeleton 100 through a user interface presented on the device 250 based on one or more applications executing on the device 250. In some cases, the device 250 can perform the operations of the robot control system 156, in whole or in part, as described herein. In that case, the device 250 can perform the operations of the robot control system 156 and direct the motor drivers 153 via data communications over the communications interface 154.

The battery 150A can be embodied as any suitable battery capable of storing power to operate the embedded controller 150, the motors 140-142, and other parts of the exoskeleton 100. As examples, the battery 150A can be embodied as one or more alkaline, lithium-ion, nickel-cadmium, nickel-metal hydride, or other types of batteries. The battery 150A can be rechargeable and, in some cases, include a recharging controller and other control circuitry to monitor the current level of charge and discharge rate of the battery 150A.

The IMUs 166-168 can be embodied as inertial measurement units capable of measuring orientation, velocity, acceleration, and other inertial information related to the exoskeleton 100. The IMUs 166-168 can provide feedback related to the motion, orientation, and position of components in the exoskeleton 100 in three-dimensional space. The IMUs 166-168 can include one or more accelerometers, gyroscopes, magnetometers, and other types of sensors to calculate inertial and position information for the exoskeleton 100. The IMU 166 is positioned at or on the attachment anchor 135 over the forearm 112. The IMU 167 is positioned at or on the housing of the motor 142 over the wrist 113. The IMU 168 is positioned at or on the attachment platform 123A over the hand 114. IMUs 166-168 can provide feedback signals or data to the embedded controller 150, with the feedback signals identifying the orientation, velocity, acceleration, and other inertial and position information for the exoskeleton 100. The embedded controller 150 can also include an additional IMU in some cases, and the exoskeleton 100 can incorporate additional IMUs at other locations. Additionally, in some cases, one or more of the IMUs 166-168 can be omitted from the exoskeleton 100.

The feedback data from the IMUs 166-168 can be stored in the data store 152 as the control information 152A for further processing by the embedded controller 150. Based in part on the feedback data from the IMUs, the embedded controller 150 can calculate the position and movement of the arm 110 over time, including the movement of the bicep 111, the forearm 112, the wrist 113, and the hand 114 of the arm 110. The embedded controller 150 can also calculate, determine, and monitor the motion of the arm 110 based on the control information 152A. The embedded controller 150 is also configured to identify, isolate, and analyze the portion of the movement in the arm 110 that is attributed to the intended or voluntary movements of the individual through a frequency or related analysis of the control information 152A. The embedded controller 150 can also calculate and determine the trajectory of the intended motion of the arm 110 based in part on the control information 152A.

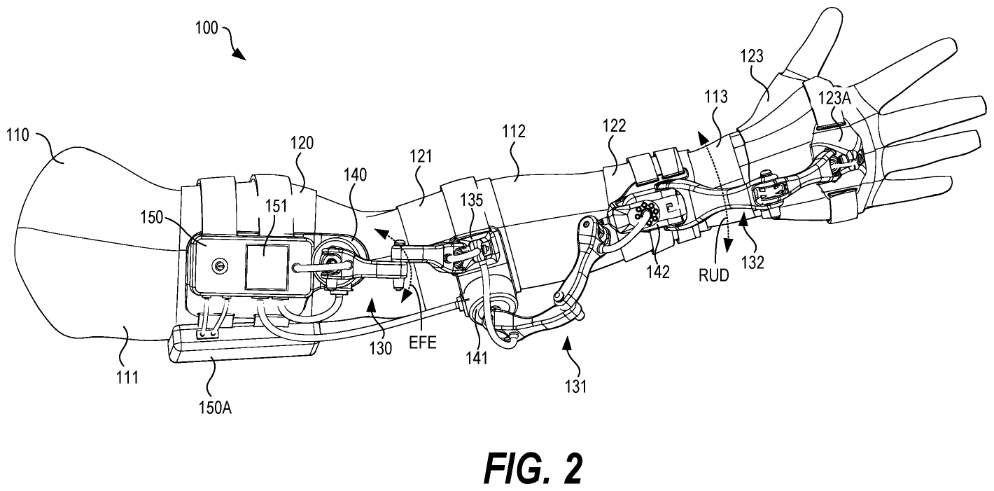

FIG. 2 illustrates a top-down view of the forearm exoskeleton 100 shown in FIG. 1 according to various embodiments described herein. The exoskeleton 100 can actively suppress tremors in four upper limb degrees of freedom (DOFs) of the arm 110. The exoskeleton 100 can actively suppress tremors for the EFE, the FPS, the WFE, and the RUD motions in the arm 110. Referring to FIG. 2, the pivotable linkage 130 extends between a joint connection at the motor 140 and a joint connection at the attachment anchor 135. The motor 140 and the kinematic chain provided between the motor 140, the pivotable linkage 130, the attachment anchor 135, and the joints therebetween can actively suppress tremors in the EFE of the arm 110. Additionally, the pivotable linkage 132 extends between a hub joint connection at the motor 142 and a joint connection at the attachment platform 123A. The motor 142 and the kinematic chain provided between the motor 142, the pivotable linkage 132, the attachment platform 123A, and the joints therebetween can actively suppress tremors in the RUD of the arm 110.

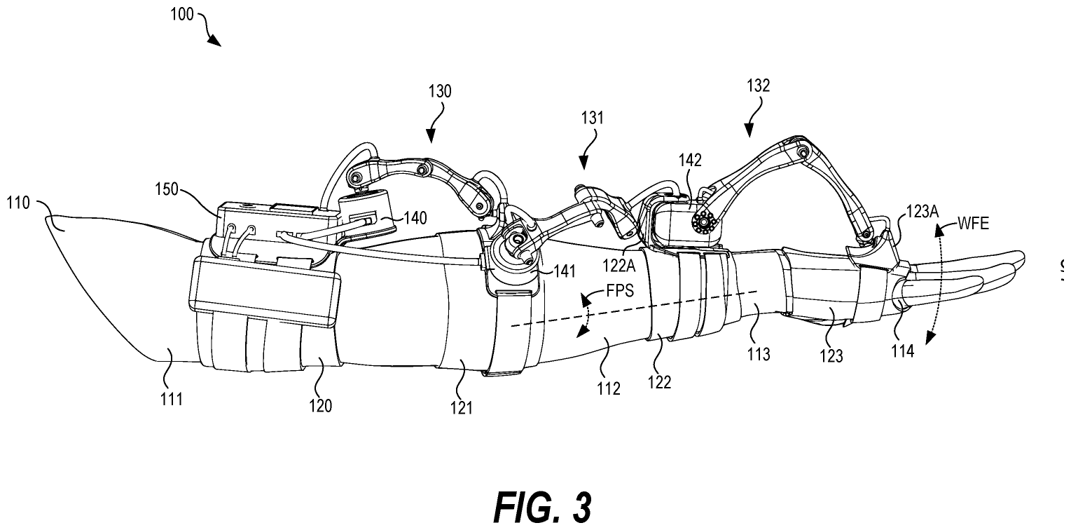

FIG. 3 illustrates a side view of the forearm exoskeleton 100 shown in FIG. 1 according to various embodiments described herein. The pivotable linkage 131 extends between a joint connection at the motor 141 and a joint connection at the attachment platform 122A. The motor 141 and the kinematic chain provided between the motor 141, the pivotable linkage 131, the attachment platform 122A, and the joints therebetween can actively suppress tremors in the FPS of the arm 110. Additionally, the pivotable linkage 132 extends between another hub joint connection at the motor 142 and a joint connection at the attachment platform 123A. The motor 142 and the kinematic chain provided between the motor 142, the pivotable linkage 132, the attachment platform 123A, and the joints therebetween can actively suppress tremors in the WFE of the arm 110.

FIG. 4 illustrates the pivotable linkage 130 and pivot points or joints 180-182 of the pivotable linkage 130 in the exoskeleton 100 shown in FIG. 1 according to various embodiments described herein. Particularly, FIG. 4 illustrates the links 130A and 130B of the pivotable linkage 130, separated from the joints 180-182. The joint 180 is positioned at an end of a shaft of the motor 140. The joint 180 provides two degrees of freedom between the motor 140 and the link 130A. The joint 181 provides one degree of freedom between the link 130A and the link 130B. The joint 182 provides three degrees of freedom between the link 130B and the attachment anchor 135. The joint 182 is a Euler-type joint, as shown in FIG. 2A. Movement (i.e., rotation) of the motor 140 imparts forces on the pivotable linkage 130 and between the attachment platform 120A and the attachment anchor 135. In turn, movement of the motor 140 imparts forces between the bicep 111 and forearm 112 of the arm 110 for EFE movement.

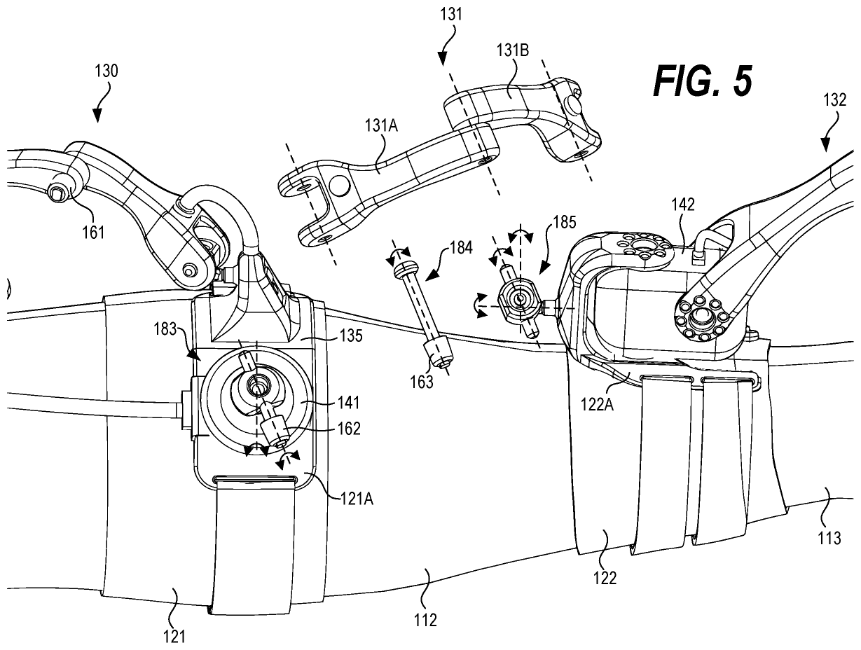

FIG. 5 illustrates the pivotable linkage 131 and pivot points or joints 183-185 of the pivotable linkage 131 in the exoskeleton 100 shown in FIG. 1 according to various embodiments described herein. Particularly, FIG. 5 illustrates the links 131A and 131B of the pivotable linkage 131, separated from the joints 183-185. The joint 183 is positioned at an end of a shaft of the motor 141. The joint 183 provides two degrees of freedom between the motor 141 and the link 131A. The joint 184 provides one degree of freedom between the link 131A and the link 131B. The joint 185 provides three degrees of freedom between the link 131B and the attachment platform 122A. The joint 185 is a Euler-type joint. Movement of the motor 141 imparts forces on the pivotable linkage 131 and between the attachment platform 121A and the attachment platform 122A. In turn, movement of the motor 141 imparts forces between the forearm 112 and the wrist 113 of the arm 110 for FPS movement.

FIG. 6 illustrates the pivotable linkage 132 and pivot points or joints 186-189 in the exoskeleton 100 shown in FIG. 1 according to various embodiments described herein. Particularly, FIG. 6 illustrates the links 132A and 132B of the pivotable linkage 132. The joint 186 is formed at a hub between the motor 142 and the attachment platform 122A and provides one degree of freedom. Particularly, the attachment platform 122A includes an attachment U-frame 125. The U-frame 125 extends up from the attachment platform 122A. The motor 142 is positioned and secured within the U-frame 125 and can pivot at the joint 186 with respect to the attachment platform 122A. More particularly, the motor 142 includes two separate motors, with one capable of pivoting the motor 142 (or the housing of the motor 142) with respect to the U-frame 125 at the joint 186. In turn, movement of the motor 142 at the joint 186 imparts forces on the wrist 113 of the arm 110 for RUD movement.

Additionally, the motor 142 is also capable of pivoting or rotating the link 132A with respect to the housing of the motor 142 at the joint 187. The joint 187 is formed at another hub between the motor 142 and the link 132A and provides one degree of freedom. Movement of the motor 142 at the joint 187 imparts forces on the link 132A and, in turn, on the hand 114 of the arm 110 for WFE movement. The joint 188 provides one degree of freedom between the link 132A and the link 132B. The joint 189 provides three degrees of freedom between the link 132B and the attachment platform 123A. The joint 189 is a Euler-type joint. The joint 189 permits additional degrees of freedom between the wrist 113 and the hand 114 while also permitting the exoskeleton to impart RUD and WFE control.

Referring among the illustrations, exoskeleton 100 includes a kinematic chain. The kinematic chain extends between the motors 140-142, the joints 180-189, the pivotable linkages 130-132, and the attachment modules 120-123. The joints 180-189 are positioned in the kinematic chain between the motors 140-142, between the motors 140-142 and the pivotable linkages 130-132, in the pivotable linkages 130-132, and between the pivotable linkages 130-132 and the attachment modules 120-123 (and the attachment anchors 135 and 136). The degrees of freedom at each of the joints 180-189, the distances between the attachment modules 120-123, the lengths of the pivotable linkages 130-132 (and the individual links in the pivotable linkages 130-132), and related mechanical aspects of the exoskeleton 100 are stored as the model parameters 152B in the embedded controller 150. Thus, the embedded controller 150 can reference the model parameters 152B to form one or more models for the kinematic chain of the exoskeleton 100. The embedded controller 150 can evaluate the models for the kinematic chain of the exoskeleton 100 as part of the control algorithm used by the robot control system 156 to direct the motors 140-142 for tremor suppression and assistance with the voluntary movement of the arm 110.

Referring to FIG. 1, the robot control system 156 includes the model identifier 157, the motion planner 158, and the motion controller 159. Among other processes, the robot control system 156 is configured to store and analyze the motion of the arm 110, in real time, based on the feedback signals and data provided from the positional encoders in the motors 140-142, the positional encoders 160-164, and the IMUs 166-168. The real-time motion of the arm 110 can be analyzed and monitored in view of the feedback data, as the feedback data is indicative of the actual movement of the arm 110 over time. The real-time motion of the arm 110 can be used by the model identifier 157 to develop models associated with the arm 110, as described below, and to recursively update the models over time. The real-time motion of the arm 110 can also be analyzed and monitored in connection with the models and model parameters 152B associated with the exoskeleton 100. The real-time motion of the arm 110 can also be used by the motion planner 158 to plan the motion of and forecast trajectories of the arm 110.

The robot control system 156 is configured to analyze the motion of the arm 110 through a frequency or related analysis of the feedback data. For an individual with Parkinson's disease, for example, the motion of the arm 110 can include both involuntary quivering movements (e.g., tremor motion) and voluntary movements. Based on the results of the frequency analysis of the feedback data and the control information 152A, the robot control system 156 can identify and isolate the portion of the movement in the arm 110 that is attributed to the involuntary quivering movements. The robot control system 156 is also configured to identify and isolate the portion of the movement in the arm 110 that is attributed to the intended or voluntary movements of the arm 110. Data related to both the tremor motion and the voluntary movements of the arm 110 can be stored in the data store 152 for further processing.

The model identifier 157 is configured to perform a multibody analysis on the exoskeleton 100 using the model parameters 152B and the real-time motion of the arm 110 with reference to the control information 152A. In some cases, the model identifier 157 can perform a multibody analysis on the exoskeleton 100 using the model parameters 152B and example data related to motions of the arm 110, rather than data related to the real-time motion of the arm 110. As part of the multibody analysis, the model identifier 157 is configured to identify the kinematic parameters of motion associated with the EFE, FPS, WFE, and RUD motions in the arm 110.

The model identifier 157 is informed of the kinematic structure of the exoskeleton 100 based on the model parameters 152B stored in the data store 152. The model identifier 157 is also informed of the motions of the exoskeleton 100 and the arm based on the control information 152A stored in the data store 152. However, the kinematic structure of the exoskeleton 100 and the arm 110 may be undefined and vary from arm to arm. Thus, the model identifier 157 is also configured to identify a range of kinematic parameters of motion that are particular to the exoskeleton 100, to the arm 110, or both for the EFE, FPS, WFE, and RUD motions in the arm 110. The model identifier 157 is also configured to develop one or more models that are particular to the EFE, FPS, WFE, and RUD motions in the exoskeleton 100, in the arm 110, or both.

For example, the model identifier 157 is configured to identify the kinematic parameters of motion in the wrist 113 and the hand 114 by performing a wrist kinematic identification (WKI) algorithm. The WKI algorithm can be performed through an ellipsoidal joint approximation model and real-time sparsity-promoting regression, for example, by the model identifier 157. The results of the WKI algorithm define additional parameters related to the kinematic structure of the wrist 113 and the hand 114 and are stored as the model parameters 152B in the data store 152. As another example, the model identifier 157 is configured to identify the kinematic parameters of motion in the wrist 113 and the forearm 112 by performing a forearm kinematic identification (FKI) algorithm. The FKI algorithm can be performed through another joint approximation model by the model identifier 157. The results of the FKI algorithm define additional parameters related to the kinematic structure of the wrist 113, the forearm 112, and the joints between them, and those parameters are also stored as the model parameters 152B in the data store 152. The model identifier 157 is also configured to identify the kinematic parameters of motion in the forearm 112 and the bicep 111 by performing an upper arm kinematic identification (UKI) algorithm. The UKI algorithm can be performed through another joint approximation model by the model identifier 157. The results of the UKI algorithm define additional parameters related to the kinematic structure of the forearm 112, the bicep 111, and the joints between them, and those parameters are stored also as the model parameters 152B in the data store 152. The WKI, FKI, UKI, and possibly other models can be developed by the model identifier 157 through the real-time analysis of the motion of the arm 110. The models can also be developed based on example or training data in some cases. The WKI, FKI, and UKI models can also be recursively updated in real time by the model identifier 157.

The motion planner 158 of the robot control system 156 is configured to perform a motion planning algorithm for the exoskeleton 100. The motion planner 158 can process the data related to both the tremor motion and the voluntary movements of the arm 110, in real time, as that data is analyzed and stored in the data store 152 by the robot control system 156. The motion planner 158 can also extrapolate or project a forecasted trajectory for the arm 110. That is, based on the isolation of voluntary movement of the arm 110 over a partial trajectory, the motion planner 158 can forecast the remainder of the trajectory (e.g., an extended or forecasted trajectory) of the arm 110 for rehabilitation or assistive purposes.

The motion planner 158 is configured to perform trajectory tracking and forecasting under inertia/load uncertainties and external disturbances for both the arm 110 and the exoskeleton 100. In one example, the motion planner 158 incorporates an adaptive controller and can perform an inverse optimality technique for motion tracking and trajectory forecasting based on a general model for upper limb rehabilitation. The motion planner 158 can also perform motion tracking and trajectory forecasting based on the previously-developed WKI, FKI, and UKI models to tailor the forecasted trajectories in view of the particular kinematic structure of the arm 110, as previously modeled by the model identifier 157. The motion planner 158 is also configured to perform trajectory forecasting in view of uncertainties in the previously-developed WKI, FKI, and UKI models.

Additionally, the motion planner 158 can also perform trajectory forecasting based on the known model parameters 152B of the exoskeleton 100 to tailor the control of the exoskeleton 100. Thus, the motion planner 158 can develop a forecasted trajectory of the arm 110 based on both models of the arm 110, as determined by the model identifier 157, and the known model parameters 152B of the exoskeleton 100. Forecasted trajectories of the arm 110, as developed by the motion planner 158, can be stored as the motion trajectories 152C in the data store 152.

The motion planner 158 can incorporate learning models or algorithms in some cases, to help estimate the trajectory of the intended motion of the arm 110. For example, data related to a history of the voluntary movements of the arm 110 can be stored in the motion trajectories 152C. As the intended motion of the arm 110 is currently isolated, in real time, by the robot control system 156, the current voluntary motion can be compared against the history of the voluntary movements of the arm 110 stored in the motion trajectories 152C. The motion planner 158 can seek to identify correlations (e.g., trajectory matches) between the current voluntary motion of the arm 110 and the history of the previous voluntary movements of the arm 110 stored in the motion trajectories 152C to develop the forecasted trajectory of the intended motion of the arm 110.

The motion controller 159 of the robot control system 156 is configured to develop motor control signals for control of the motors 140-142. In one example, the motion controller 159 is configured to control the motors 140-142 in the exoskeleton 100 for two primary objectives, including tremor suppression and voluntary movement assistance. For example, based on the results of the frequency analysis performed by the robot control system 156, the robot control system 156 can isolate the portion of the movement in the arm 110 that is attributed to the involuntary quivering movements of the arm 110. The robot control system 156 can also isolate the portion of the movement in the arm 110 that is attributed to the intended or voluntary movements of the arm 110. Additionally, the motion planner 158 can evaluate the trajectory, or partial trajectory, of the intended motion of the arm 110. The motion planner 158 can also forecast, in some cases, a remaining or extended trajectory of the arm 110. The motion controller 159 can also control the motors 140-142 for other objectives, such as for teaching or rehabilitative motions.

In view of the isolated voluntary and involuntary components of movement in the arm 110, the motion controller 159 is configured to develop motor control signals that suppress (e.g., counterbalance against) the involuntary quivering movements of the arm 110 and enhance (e.g., support or further direct) the intended or voluntary movements of the arm 110. The motion controller 159 can develop the motor control signals for enhancing the intended movements of the arm 110 based on the forecasted trajectory of the intended motion of the arm 110, as provided by the motion planner 158. The motion controller 159 can also control the motors 140-142 for other objectives, however, such as to teach an individual new motions or new motion trajectories for rehabilitative purposes.

In some aspects, the motion controller 159 is configured to develop motor control signals through the minimization of interaction resistance between an individual and the exoskeleton 100. Based on model assumptions, the motion controller 159 can optimize the tracking reference of the exoskeleton 100 by minimizing the human-exoskeleton interaction loads estimated, e.g., based on a support vector regression (SVR) model. The model can also be updated recursively by the robot control system 156 in real time.

The motion control signals are provided to the motor drivers 153 to control the motors 140-142. Based on the motor control signals, the motors 140-142 can impart forces on the exoskeleton 100 to both suppress tremors and to assist with voluntary movements in the arm 110. Through the signal processing performed by the robot control system 156, the motors 140-142 produce safe actuations based on adaptive control and motion planning algorithms to simultaneously suppress tremors and assist with voluntary motions. The exoskeleton 100 provides a non-invasive, compliant, and stable tremor suppression and rehabilitation system for the arm 110. The exoskeleton 100 also provides movement assistance in the arm 110. Beyond healthcare, the exoskeleton 100 can also be applied to other fields and uses, such as to reduce fatigue in manual laborers tasked with repetitive and dexterous task through continual assistance.

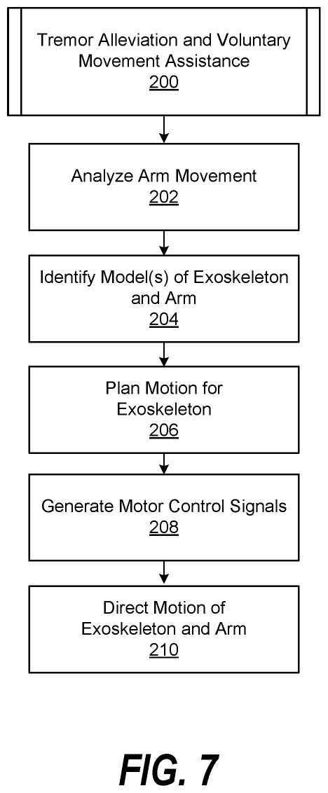

Turning to other examples, FIG. 7 illustrates an example method 200 of tremor alleviation and voluntary movement assistance using an exoskeleton according to various embodiments described herein. Although the method or process shown in FIG. 7 is described in connection with the exoskeleton 100 shown in FIG. 1, the method or process can be performed by other exoskeletons or assistive systems. Additionally, although the process is described to occur in a certain sequence, the process steps can be varied as compared to that shown. One or more of the process steps can be performed in alternative sequences or orders, concurrently, with partial concurrence, or in other ways. One or more of the process steps can be omitted, and additional steps can be added in some cases.

At step 202, the process includes analyzing movement data of an exoskeleton based on position feedback from the exoskeleton. For example, step 202 can include the robot control system 156 receiving feedback signals and data from the positional encoders in the motors 140-142, the positional encoders 160-164, and the IMUs 166-168 of the exoskeleton 100. The robot control system 156 can store data related to the feedback signals as the control information 152A in the data store 152 of the embedded controller 150. Step 202 also includes analyzing the movement data of the arm 110 through frequency analysis techniques. For example, step 202 includes the robot control system 156 analyzing the motion of the arm 110 through a frequency analysis and identifying both involuntary and voluntary movements. Step 202 also includes the robot control system 156 identifying and isolating the portion of the movement in the arm 110 that is attributed to the involuntary quivering movements. It also includes identifying and isolating the portion of the movement in the arm 110 that is attributed to the intended or voluntary movements of the arm 110. Data related to both the involuntary and voluntary movements of the arm 110 can be stored in the data store 152 for further processing at step 202 and in later process steps.

At step 204, the process includes identifying or generating one or more models based on the analysis at step 202. For example, step 204 can include the model identifier 157 performing a multibody analysis on the exoskeleton 100, on the arm 110, or on both using the model parameters 152B and the analysis of the motion of the arm 110 from step 202. As part of the multibody analysis, step 204 includes the model identifier 157 identifying the kinematic parameters of motion associated with the EFE, FPS, WFE, and RUD motions in the arm 110, as described above, and performing one or more of the WKI, FKI, and UKI algorithms to develop the models of the arm 110, develop models of the exoskeleton 100, or both. The models can be developed by the model identifier 157 through the real-time analysis of the motion of the arm 110. The models can also be developed based on example or training data in some cases. The models can also be updated recursively in real time by the model identifier 157 at step 204.

At step 206, the process includes planning motion for the arm 110 and the exoskeleton 100. For example, step 206 can include the motion planner 158 processing the involuntary and voluntary movements of the arm 110, as isolated and analyzed at step 202, using the models identified at step 204. Step 206 can also include the motion planner 158 extrapolating or projecting a forecasted trajectory for the arm 110. That is, based on the isolation of voluntary movement of the arm 110 over a partial trajectory, the motion planner 158 can forecast the remainder of the trajectory of the arm 110 for rehabilitation or assistive purposes.

The motion planner 158 can project or forecast a trajectory for the arm 110 using learning models or algorithms in some cases. For example, at step 206, the process can include the motion planner 158 identifying correlations (e.g., trajectory matches) between the current voluntary motion of the arm 110 and a history of previous voluntary movements of the arm 110 stored in the motion trajectories 152C, to develop a forecasted trajectory for the arm 110.

At step 208, the process includes generating motor control signals. For example, step 208 can include the motion controller 159 generating motor control signals for the motors 140-142. The motion controller 159 can control the motors 140-142 for two primary objectives, including tremor suppression and voluntary movement assistance. For example, based on the results of the frequency analysis performed by the robot control system 156, the robot control system 156 can isolate the portion of the movement in the arm 110 that is attributed to the involuntary quivering movements of the arm 110. The robot control system 156 can also isolate the portion of the movement in the arm 110 that is attributed to the intended or voluntary movements of the arm 110. Additionally, the motion planner 158 can evaluate the trajectory, or partial trajectory, of the intended motion of the arm 110. The motion planner 158 can also forecast, in some cases, the remaining trajectory of the arm 110.

In view of those isolated components of movement in the arm 110, step 208 can include the motion controller 159 generating motor control signals that suppress (e.g., counterbalance against) the involuntary quivering movements of the arm 110 and enhance (e.g., support or further direct) the intended or voluntary movements of the arm 110. The motion controller 159 can develop the motor control signals for enhancing the intended movements of the arm 110 based on the forecasted trajectory of the intended motion of the arm 110, as provided by the motion planner 158. The motion controller 159 can also control the motors 140-142 for other objectives, however, such as to teach an individual new motions or new motion trajectories for rehabilitative purposes.

At step 210, the process includes directing the motion of the exoskeleton 100 and the arm 110 based on the control signals generated at step 208. For example, step 210 can include the motion controller 159 providing the motion control signals to the motor drivers 153, to control the motors 140-142. Based on the motor control signals, the motors 140-142 can impart forces on the exoskeleton 100 to both suppress tremors and to assist with voluntary movements in the arm 110. Through the signal processing performed by the robot control system 156, the motors 140-142 produce safe actuations based on adaptive control and motion planning algorithms to simultaneously suppress tremors and assist with voluntary motions.

The components of the exoskeleton 100 can be embodied in hardware, software, or a combination of hardware and software. Particularly, the embedded controller 150 can be embodied in hardware, software, or a combination of hardware and software. If embodied in software, each element of the robot control system 156 can represent a module of code or a portion of code that includes program instructions to implement the specified logical function(s). The program instructions can be embodied in the form of, for example, source code that includes human-readable statements written in a programming language or machine code that includes machine instructions recognizable by a suitable execution system, such as a processor in a computer system or other system. If embodied in hardware, each element can represent a circuit or a number of interconnected circuits that implement the specified logical function(s).

The embedded controller 150 can include at least one processing circuit. Such a processing circuit can include, for example, one or more processors and one or more storage or memory devices that are coupled to a local interface. The local interface can include, for example, a data bus with an accompanying address/control bus or any other suitable bus structure. The storage devices can store data, such as the control information 152A, the model parameters 152B, and the motion trajectories 152C, as well as components that are executable by the processors of the embedded controller 150. For example, the robot control system 156 can be embodied as software stored in the storage devices and be executable by the processors of the embedded controller 150.

Overall, the robot control system 156 can be embodied in the form of hardware, as software components that are executable by hardware, or as a combination of software and hardware. If embodied as hardware, the robot control system 156 and other components described herein can be implemented as a circuit or state machine that employs any suitable hardware technology. The hardware technology can include, for example, one or more microprocessors, discrete logic circuits having logic gates for implementing various logic functions upon an application of one or more data signals, application specific integrated circuits (ASICs) having appropriate logic gates, programmable logic devices (e.g., field-programmable gate array (FPGAs), and complex programmable logic devices (CPLDs)).

Also, one or more or more of the components described herein that include software or program instructions can be embodied in a non-transitory computer-readable medium, such as a memory device or memory devices, for use by an instruction execution system, such as a processor. The computer-readable medium can contain, store, and/or maintain the software or program instructions for use by or in connection with the instruction execution system. The computer-readable medium can be embodied as a physical media, such as magnetic, optical, semiconductor, and/or other suitable media. Examples of a suitable computer-readable media include, but are not limited to, solid-state drives, magnetic drives, or flash memory. Further, any logic or component described herein can be implemented and structured in a variety of ways. For example, one or more components described can be implemented as modules or components of a single application. Further, one or more components described herein can be executed in one computing device or by using multiple computing devices.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A forearm exoskeleton for an arm, comprising:
a plurality of attachment modules;
a plurality of pivotable linkages between the plurality of attachment modules,
a plurality of motors mechanically coupled between the plurality of attachment modules and the plurality of pivotable linkages;
feedback sensors that provide position feedback for the exoskeleton; and
a controller configured to direct the plurality of motors to suppress tremors in and assist with voluntary motions of the arm based on the position feedback.

2. The forearm exoskeleton according to claim 1, wherein each of the plurality of pivotable linkages comprises a first link, a second link, a pivot point between the first link and the second link, and a position encoder at the pivot point.

3. The forearm exoskeleton according to claim 1, wherein the controller is further configured to direct the plurality of motors based on a motion planning algorithm using the position feedback.

4. The forearm exoskeleton according to claim 1, wherein the controller is further configured to develop a model for the arm using the position feedback.

5. The forearm exoskeleton according to claim 1, wherein the controller is further configured to:
develop a model for the arm using the position feedback; and
direct the plurality of motors based on a motion planning algorithm using the position feedback and the model for the arm.

6. The forearm exoskeleton according to claim 1, wherein the controller is further configured to isolate involuntary movement from voluntary movement in the arm using the position feedback.

7. The forearm exoskeleton according to claim 6, wherein the controller is further configured to direct the plurality of motors based on a motion planning algorithm to suppress the involuntary movement and assist the voluntary movement in the arm.

8. The forearm exoskeleton according to claim 1, wherein the feedback sensors comprise at least one inertial measurement unit, at least one positional encoder in at least one of the plurality of motors, and at least one positional encoder in at least one of the plurality of pivotable linkages.

9. The forearm exoskeleton according to claim 1, wherein at least one pivotable linkage among the plurality of pivotable linkages comprises an Euler-type joint.

10. The forearm exoskeleton according to claim 1, wherein the plurality of attachment modules comprise an attachment module for a bicep of the arm, an attachment module for a proximal end of a forearm of the arm, an attachment module for a distal end of the forearm, and an attachment module for a dorsum of a hand of the arm.

11. The forearm exoskeleton according to claim 1, wherein the exoskeleton provides passive and active suppression of tremors in the arm.

12. The forearm exoskeleton according to claim 1, wherein the controller is further configured to:
perform a first kinematic identification algorithm for elbow flexion-extension (EFE) motion of the arm using the position feedback;
perform a second kinematic identification algorithm for forearm pronation-supination (FPS) motion of the arm using the position feedback;
perform a third kinematic identification algorithm for wrist flexion-extension (WFE) motion of the arm using the position feedback; and
perform a fourth kinematic identification algorithm for wrist radial-ulnar deviations (RUD) motions of the arm using the position feedback.

13. The forearm exoskeleton according to claim 12, wherein the controller is further configured to:
develop a model for the arm based on a combination of the first, the second, the third, and the fourth kinematic identification algorithms; and
direct the plurality of motors based on the model for the arm using the position feedback.

14. The forearm exoskeleton according to claim 13, wherein the controller is further configured to recursively update the model for the arm.

15. The forearm exoskeleton according to claim 1, wherein the controller is further configured to:
perform trajectory tracking of an intended motion of the arm based on the position feedback;
forecast a trajectory of the intended motion of the arm based on the position feedback; and
direct the plurality of motors based on a motion planning algorithm to suppress involuntary movement in the arm and to assist with voluntary movement in the arm based on the forecast of the trajectory of the intended motion.

16. The forearm exoskeleton according to claim 1, wherein the controller is further configured to minimize human-exoskeleton interaction loads estimated based on a real-time updated regression model.

17. A method for control of a forearm exoskeleton for an arm, comprising:
analyzing movement data of the forearm exoskeleton based on position feedback from feedback sensors of the forearm exoskeleton;
generating a model for the forearm exoskeleton based on the analyzing;
planning motion for the arm and the exoskeleton based on the model; and
directing at least one motor of the forearm exoskeleton based on the planning.

18. The method according to claim 17, wherein the forearm exoskeleton comprises:
a plurality of attachment modules;
a plurality of pivotable linkages between the plurality of attachment modules,
a plurality of motors mechanically coupled between the plurality of attachment modules and the plurality of pivotable linkages;
feedback sensors that provide the position feedback for the exoskeleton; and
a controller.

19. The method according to claim 18, wherein the feedback sensors comprise at least one inertial measurement unit and at least one positional encoder.

20. The method according to claim 18, wherein the plurality of attachment modules comprise an attachment module for a bicep of the arm, an attachment module for a proximal end of a forearm of the arm, an attachment module for a distal end of the forearm, and an attachment module for a dorsum of a hand of the arm.
